(19) United States
(12) Patent Application Publication
(10) Pub. No.: US 2011/0130326 A1
(43) Pub. Date: Jun. 2, 2011

(54) DEFENSINS FOR TREATMENT OF INFECTIVE ENDOCARDITIS

(75) Inventor: Hans-Henrik Kristensen Hoeghenhaug, Holte (DK)

(73) Assignee: Novozymes A/S, Bagsvaerd (DK)

(21) Appl. No.: 12/956,577

(22) Filed: Nov. 30, 2010

Related U.S. Application Data
(60) Provisional application No. 61/332,877, filed on May 10, 2010.
(60) Provisional application No. 61/267,132, filed on Dec. 7, 2009.

(30) Foreign Application Priority Data
Dec. 2, 2009 (EP) 09 177 773.0
May 7, 2010 (EP) 10 162 348.6

(51) Int. Cl. A61K 38/16; A61P 31/04; A61P 9/00

(52) U.S. Cl. 514/2.7; 514/2.3

(57) ABSTRACT

The present invention relates to methods for treating infective endocarditis, such as bacterial endocarditis, with defensin polypeptides.

### CROSS-REFERENCE TO RELATED APPLICATIONS

This application claims priority or the benefit under 35 U.S.C. 119 of European application nos. 10 162 348.6 filed May 7, 2010 and 09 177 773.0 filed Dec. 2, 2009, and U.S. provisional application Nos. 61/332,877 filed May 10, 2010 and 61/267,132 filed Dec. 7, 2009, the contents of which are fully incorporated herein by reference.

### REFERENCE TO A SEQUENCE LISTING

This application contains a Sequence Listing in computer readable form, which is incorporated herein by reference.

### BACKGROUND OF THE INVENTION

1. Field of the Invention

The present invention relates to the treatment of infective endocarditis with defensin polypeptides.

2. Background

Endocarditis is an inflammation of the inner layer of the heart, the endocardium. It usually involves the heart valves (native or prosthetic valves). Other structures which may be involved include the interventricular septum, the chordae tendinae, the mural endocardium, or even on intracardiac devices. Endocarditis is characterized by a prototypic lesion, the vegetation, which is a mass of platelets, fibrin, microcolonies of microorganisms, and scant inflammatory cells.

There are multiple ways to classify endocarditis. The simplest classification is based on etiology: either infective or non-infective, depending on whether a microorganism is the source of the inflammation. Regardless, diagnosis of endocarditis is based on the clinical features, investigations such as echocardiogram, as well as any blood cultures demonstrating the presence of endocarditis-causing microorganisms.

Since the valves of the heart do not receive any dedicated blood supply, drugs have difficulty reaching the infected valve via the bloodstream to cure infective endocarditis (IE). In particular, methicilin-resistant Staphylococcus aureus (MRSA) caused infective endocarditis is very difficult to treat.

According to Fowler et al., 2005, “Staphylococcus aureus endocarditis: A consequence of medical progress”, J. Am. Med. Assoc. 293(24): 3012-3021 (2005), Staphylococcus aureus was the most common pathogen among 1779 cases of definite IE in the International Collaboration on Endocarditis Prospective-Cohort Study (558 patients, 31.4%). MRSA IE was more common in the United States (37.2%) and Brazil (37.5%) than in Europe/Middle East (23.7%).

It is an object of the present invention to provide polypeptides, which can be used for the treatment of infective endocarditis.

### SUMMARY OF THE INVENTION

We have now found that a synthetic defensin antimicrobial peptide shows excellent activity against endocarditis, and can be used in the treatment of infective endocarditis.

In a first aspect, the present invention provides the use of a polypeptide having antimicrobial activity, which comprises an amino acid sequence having at least 80% identity to the amino acid sequence of SEQ ID NO: 1, for the manufacturing of a medicament for therapeutic treatment of infective endocarditis.

In a second aspect, the present invention provides a polypeptide having antimicrobial activity, which comprises an amino acid sequence having at least 80% identity to the amino acid sequence of SEQ ID NO: 1, for use in the treatment of infective endocarditis.

In another aspect the present invention provides a method of treating infective endocarditis, comprising administering to a subject in need of such treatment an effective amount of a polypeptide having antimicrobial activity, which comprises an amino acid sequence having at least 80% identity to the amino acid sequence of SEQ ID NO: 1.

In one embodiment, the polypeptide is a defensin polypeptide, preferably a beta-defensin polypeptide.

Infective endocarditis according to the present invention may be a bacterial endocarditis, preferably a staphylococcal endocarditis. In a preferred embodiment, the infective endocarditis is caused by methicilin-resistant Staphylococcus aureus (MRSA).

A polypeptide for use according to the present invention, or for treating infective endocarditis according to the present invention, is designated hereinafter as “polypeptide(s) of (according to) the present invention”.

### DETAILED DESCRIPTION OF THE INVENTION

### Definitions

Antimicrobial activity: The term “antimicrobial activity” is defined herein as an activity which is capable of killing or inhibiting growth of microbial cells. In the context of the present invention the term “antimicrobial” is intended to mean that there is a bactericidal and/or a bacteriostatic and/or fungicidal and/or fungistatic effect, wherein the term “bactericidal” is to be understood as capable of killing bacterial cells. The term “bacteriostatic” is to be understood as capable of inhibiting bacterial growth, i.e., inhibiting growing bacterial cells. The term “fungicidal” is to be understood as capable of killing fungal cells. The term “fungistatic” is to be understood as capable of inhibiting fungal growth, i.e., inhibiting growing fungal cells. The term “microbial cells” denotes bacterial or fungal cells (including yeasts).

In the context of the present invention the term “inhibiting growth of microbial cells” is intended to mean that the cells are in the non-growing state, i.e., that they are not able to propagate.

In a preferred embodiment, the term “antimicrobial activity” is defined as bactericidal and/or bacteriostatic activity. More preferably, “antimicrobial activity” is defined as bactericidal and/or bacteriostatic activity against Streptococci, preferably Streptococcus pneumoniae.

For purposes of the present invention, antimicrobial activity may be determined according to the procedure described by Lehrer et al., 1991, Journal of Immunological Methods 137(2): 167-174. Alternatively, antimicrobial activity may be determined according to the NCCLS guidelines from CLSI (Clinical and Laboratory Standards Institute; formerly known as National Committee for Clinical and Laboratory Standards).

Polypeptides having antimicrobial activity may be capable of reducing the number of living cells of Staphylococcus aureus (ATCC 29213) to 1/100 after 8 hours (preferably after 4 hours, more preferably after 2 hours, most preferably after 1 hour, and in particular after 30 minutes) incubation at 37° C. in a relevant microbial growth substrate at a concentration of 500 μg/ml; preferably at a concentration of 250 μg/ml; more preferably at a concentration of 100 μg/ml; even more preferably at a concentration of 50 μg/ml; most preferably at a concentration of 25 μg/ml; and in particular at a concentration of 10 μg/ml of the polypeptides having antimicrobial activity.

Polypeptides having antimicrobial activity may also be capable of inhibiting the outgrowth of Staphylococcus aureus (ATCC 29213) for 8 hours at 37° C. in a relevant microbial growth substrate, when added in a concentration of 500 μg/ml; preferably when added in a concentration of 250 μg/ml; more preferably when added in a concentration of 100 μg/ml; even more preferably when added in a concentration of 50 μg/ml; most preferably when added in a concentration of 10 μg/ml; and in particular when added in a concentration of 5 μg/ml.

The polypeptides of the present invention have at least 20%, preferably at least 40%, more preferably at least 50%, more preferably at least 60%, more preferably at least 70%, more preferably at least 80%, even more preferably at least 90%, most preferably at least 95%, and even most preferably at least 100% of the antimicrobial activity of the polypeptide consisting of the amino acid sequence of SEQ ID NO: 1.

Defensin: The term “defensin” as used herein refers to polypeptides recognized by a person skilled in the art as belonging to the defensin class of antimicrobial peptides. To determine if a polypeptide is a defensin according to the invention, the amino acid sequence is preferably compared with the hidden markov model profiles (HMM profiles) of the PFAM database by using the freely available HMMER software package.

The PFAM defensin families include Defensin_{—}1 or “Mammalian defensin” (accession no. PF00323), Defensin_{—}2 or “Arthropod defensin” (accession no. PF01097), Defensin_beta or “Beta Defensin” (accession no. PF00711), Defensin_propep or “Defensin propeptide” (accession no. PF00879) and Gamma-thionin or “Gamma-thionins family” (accession no. PF00304).

The defensins may belong to the alpha-defensin class, the beta-defensin class, the theta-defensin class, the insect or arthropod defensin classes, or the plant defensin class.

In an embodiment, the amino acid sequence of a defensin according to the invention comprises 4, 6 or 8 cysteine residues, preferably 4 or 6 cysteine residues, more preferably 6 cysteine residues.

The defensins may also be synthetic defensins sharing the characteristic features of any of the defensin classes.

Isolated polypeptide: The term “isolated variant” or “isolated polypeptide” as used herein refers to a variant or a polypeptide that is isolated from a source. In one aspect, the variant or polypeptide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by SDS-PAGE.

Substantially pure polypeptide: The term “substantially pure polypeptide” denotes herein a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which it is natively or recombinantly associated. It is, therefore, preferred that the substantially pure polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The polypeptides of the present invention are preferably in a substantially pure form. This can be accomplished, for example, by preparing the polypeptide by well-known recombinant methods or by classical purification methods.

Identity: The relatedness between two amino acid sequences or between two nucleotide sequences is described by the parameter “identity”.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; http://emboss.org), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra; http://emboss.org), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

Allelic variant: The term “allelic variant” denotes herein any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Modification: The term “modification” means herein any chemical modification of the polypeptide consisting of the amino acid sequence of SEQ ID NO: 1 as well as genetic manipulation of the DNA encoding that polypeptide. The modification(s) can be substitution(s), deletion(s) and/or insertions(s) of the amino acid(s) as well as replacement(s) of amino acid side chain(s); or use of unnatural amino acids with similar characteristics in the amino acid sequence. In particular the modification(s) can be amidations, such as amidation of the C-terminus.

### Polypeptides Having Antimicrobial Activity

In a first aspect, the present invention relates to isolated polypeptides having an amino acid sequence which has a degree of identity to SEQ ID NO: 1 (i.e., the mature polypeptides) of at least 80%, preferably at least 85%, more preferably at least 90%, most preferably at least 95%, and in particular at least 97%, which have antimicrobial activity (hereinafter “homologous polypeptides”). In a preferred aspect, the homologous polypeptides have an amino acid sequence which differs by at the most eight amino acids, preferably by at the most seven amino acids, more preferably by at the most six amino acids, even more preferably by at the most five amino acids, even more preferably by at the most four amino acids, even more preferably by at the most three amino acids, most preferably by at the most two amino acids, and in particular by one amino acid from the amino acid sequence of SEQ ID NO: 1.

In yet another aspect, the polypeptides of the invention has one or several amino acid changes compared to the amino acid sequence of SEQ ID NO: 1.

A polypeptide of the present invention preferably comprises the amino acid sequence of SEQ ID NO: 1 or an allelic variant thereof. In a preferred aspect, a polypeptide comprises the amino acid sequence of SEQ ID NO: 1. In another preferred aspect, a polypeptide consists of the amino acid sequence of SEQ ID NO: 1 or an allelic variant thereof. In another preferred aspect, a polypeptide consists of the amino acid sequence of SEQ ID NO: 1.

Preferably, amino acid changes are of a minor nature, that is conservative amino acid substitutions or insertions that do not significantly affect the folding and/or activity of the polypeptide; single deletions; small amino- or carboxyl-terminal extensions; a small linker peptide of up to about 20-25 residues; or a small extension that facilitates purification by changing net charge or another function, such as a poly-histidine tag, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Essential amino acids in the parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., antimicrobial activity) to identify amino acid residues that are critical to the activity of the molecule. See also, Hilton et al., 1996, J. Biol. Chem. 271: 4699-4708. The biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309:59-64. The identities of essential amino acids can also be inferred from analysis of identities with polypeptides which are related to a polypeptide according to the invention.

Single or multiple amino acid substitutions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells. Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

In a preferred embodiment, the polypeptides of the invention are defensin polypeptides, preferably beta-defensin polypeptides.

### N-Terminal Extension

An N-terminal extension of the polypeptides of the invention may suitably consist of from 1 to 50 amino acids, preferably 2-20 amino acids, especially 3-15 amino acids. In one embodiment N-terminal peptide extension does not contain an Arg (R). In another embodiment the N-terminal extension comprises a kex2 or kex2-like cleavage site as will be defined further below. In a preferred embodiment the N-terminal extension is a peptide, comprising at least two Glu (E) and/or Asp (D) amino acid residues, such as an N-terminal extension comprising one of the following sequences: EAE, EE, DE and DD.

### Kex2 Sites

Kex2 sites (see, e.g., Methods in Enzymology Vol 185, ed. D. Goeddel, Academic Press Inc. (1990), San Diego, Calif., “Gene Expression Technology”) and kex2-like sites are di-basic recognition sites (i.e., cleavage sites) found between the pro-peptide encoding region and the mature region of some proteins.

Insertion of a kex2 site or a kex2-like site have in certain cases been shown to improve correct endopeptidase processing at the pro-peptide cleavage site resulting in increased protein secretion levels.

In the context of the invention insertion of a kex2 or kex2-like site result in the possibility to obtain cleavage at a certain position in the N-terminal extension resulting in an antimicrobial polypeptide being extended in comparison to the mature polypeptide shown in SEQ ID NO: 1.

### Fused Polypeptides

The polypeptides of the present invention also include fused polypeptides or cleavable fusion polypeptides in which another polypeptide is fused at the N-terminus or the C-terminus of the polypeptide of the invention or a fragment thereof. A fused polypeptide is produced by fusing a nucleotide sequence (or a portion thereof) encoding another polypeptide to a nucleotide sequence (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator.

### Methods and Uses

The invention relates to the use of a polypeptide of the invention for treating infective endocarditis. Accordingly, the polypeptides of the invention may be used as a veterinarian or human therapeutic or prophylactic agent. Thus, polypeptides of the invention may be used for the manufacturing of a medicament for the treatment of infective endocarditis, such as bacterial endocarditis, for example Staphylococcal endocarditis or Staphylococcus aureus endocarditis. In an embodiment, infective endocarditis is caused by infection with a methicilin-resistant Staphylococcus aureus (MRSA).

The polypeptides of the invention are not only effective in treating infective endocarditis, but are also effective in preventing relapse after treatment of infective endocarditis. The invention thus provides both method and use of the polypeptides of the invention as a medicament for preventing relapse after treatment of infective endocarditis.

The polypeptides of the invention may be used in an amount sufficient to kill or inhibit growth of Staphylococcus sp., such as Staphylococcus aureus.

Formulations of the polypeptides of the invention are administered to a host suffering from or predisposed to infective endocarditis.

Administration may be localized or systemic. Generally the dose of the antimicrobial polypeptides of the invention will be sufficient to decrease the microbial population by at least 1 log, and may be by 2 or more logs of killing. The polypeptides of the present invention are administered at a dosage that reduces the microbial population while minimizing any side-effects. It is contemplated that the composition will be obtained and used under the guidance of a physician for in vivo use.

Various methods for administration may be employed. The polypeptide formulation may be given orally, or may be injected intravascularly, intramuscular, subcutaneously, peritoneally, by aerosol, opthalmically, intra-bladder, topically, etc. The dosage of the therapeutic formulation will vary widely, depending on the specific antimicrobial polypeptide to be administered, the frequency of administration, the manner of administration, the clearance of the agent from the host, and the like. The initial dose may be larger, followed by smaller maintenance doses. In many cases, oral administration will require a higher dose than if administered intravenously. The amide bonds, as well as the amino and carboxy termini, may be modified for greater stability on oral administration. For example, the carboxy terminus may be amidated.

### Formulations

The polypeptides of this invention can be incorporated into a variety of formulations for therapeutic administration. More particularly, the polypeptides of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, creams, foams, solutions, suppositories, injections, inhalants, gels, microspheres, lotions, and aerosols. As such, administration of the polypeptides can be achieved in various ways, including oral, buccal, rectal, parenteral, intraperitoneal, intradermal, transdermal, intracheal, etc., administration. The antimicrobial polypeptides of the invention may be systemic after administration or may be localized.

The polypeptides of the present invention can be administered alone, in combination with each other, or they can be used in combination with other known compounds (e.g., perforin, anti-inflammatory agents, antibiotics, etc.) In pharmaceutical dosage forms, the polypeptides may be administered in the form of their pharmaceutically acceptable salts. The following methods and excipients are merely exemplary and are in no way limiting.

For oral preparations, the polypeptides can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The polypeptides can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The polypeptides can be utilized in aerosol formulation to be administered via inhalation. The polypeptides of the present invention can be formulated into pressurized acceptable propellants such as dichlorodifluoromethane, propane, nitrogen and the like.

Furthermore, the polypeptides can be made into suppositories by mixing with a variety of bases such as emulsifying bases or water-soluble bases. The polypeptides of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more polypeptides of the present invention. Similarly, unit dosage forms for injection or intravenous administration may comprise the polypeptide of the present invention in a composition as a solution in sterile water, normal saline or another pharmaceutically acceptable carrier.

Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant containing the antimicrobial polypeptides of the invention is placed in proximity to the site of infection, so that the local concentration of active agent is increased relative to the rest of the body.

The term “unit dosage form”, as used herein, refers to physically discrete units suitable as unitary dosages for human and animal subjects, each unit containing a predetermined quantity of polypeptides of the present invention calculated in an amount sufficient to produce the desired effect in association with a pharmaceutically acceptable diluent, carrier or vehicle. The specifications for the unit dosage forms of the present invention depend on the particular polypeptide employed and the effect to be achieved, and the pharmacodynamics associated with the polypeptide in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 pg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific polypeptide, the severity of the symptoms and the susceptibility of the subject to side effects. Some of the specific polypeptides are more potent than others. Preferred dosages for a given polypeptide are readily determinable by those of skill in the art by a variety of means. A preferred means is to measure the physiological potency of a given polypeptide.

The use of liposomes as a delivery vehicle is one method of interest. The liposomes fuse with the cells of the target site and deliver the contents of the lumen intracellularly. The liposomes are maintained in contact with the cells for sufficient time for fusion, using various means to maintain contact, such as isolation, binding agents, and the like. In one aspect of the invention, liposomes are designed to be aerosolized for pulmonary administration. Liposomes may be prepared with purified proteins or peptides that mediate fusion of membranes, such as Sendai virus or influenza virus, etc. The lipids may be any useful combination of known liposome forming lipids, including cationic or zwitterionic lipids, such as phosphatidylcholine. The remaining lipid will be normally be neutral or acidic lipids, such as cholesterol, phosphatidyl serine, phosphatidyl glycerol, and the like.

For preparing the liposomes, the procedure described by Kato et al., 1991, J. Biol. Chem. 266: 3361 may be used. Briefly, the lipids and lumen composition containing peptides are combined in an appropriate aqueous medium, conveniently a saline medium where the total solids will be in the range of about 1-10 weight percent. After intense agitation for short periods of time, from about 5-60 sec., the tube is placed in a warm water bath, from about 25-40° C. and this cycle repeated from about 5-10 times. The composition is then sonicated for a convenient period of time, generally from about 1-10 sec. and may be further agitated by vortexing. The volume is then expanded by adding aqueous medium, generally increasing the volume by about from 1-2 fold, followed by shaking and cooling. This method allows for the incorporation into the lumen of high molecular weight molecules.

Formulations with Other Active Agents

For use in the subject methods, the antimicrobial polypeptides of the invention may be formulated with other pharmaceutically active agents, particularly other antimicrobial agents. Other agents of interest include a wide variety of antibiotics, as known in the art. Classes of antibiotics include penicillins, e.g., penicillin G, penicillin V, methicillin, oxacillin, carbenicillin, nafcillin, ampicillin, etc.; penicillins in combination with beta-lactamase inhibitors, cephalosporins, e.g., cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc.

Anti-mycotic agents are also useful, including polyenes, e.g., amphotericin B, nystatin; 5-flucosyn; and azoles, e.g., miconazol, ketoconazol, itraconazol and fluconazol. Antituberculotic drugs include isoniazid, ethambutol, streptomycin and rifampin. Cytokines may also be included in a formulation of the antimicrobial polypeptides of the invention, e.g., interferon gamma, tumor necrosis factor alpha, interleukin 12, etc.

### In Vitro Synthesis

The polypeptides of the invention may be prepared by in vitro synthesis, using conventional methods as known in the art. Various commercial synthetic apparatuses are available, for example automated synthesizers by Applied Biosystems Inc., Beckman, etc. By using synthesizers, naturally occurring amino acids may be substituted with unnatural amino acids, particularly D-isomers (or D-forms), e.g., D-alanine and D-isoleucine, diastereoisomers, side chains having different lengths or functionalities, and the like. The particular sequence and the manner of preparation will be determined by convenience, economics, purity required, and the like.

Chemical linking may be provided to various peptides or proteins comprising convenient functionalities for bonding, such as amino groups for amide or substituted amine formation, e.g., reductive amination, thiol groups for thioether or disulfide formation, carboxyl groups for amide formation, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein

The present invention is further described by the following examples that should not be construed as limiting the scope of the invention.

### EXAMPLES

The synthetic defensin used in the experiments is a polypeptide having the amino acid sequence shown in SEQ ID NO: 1. In the Examples, this synthetic defensin will be referred to as “endocardisin”.

### Example 1

### Therapeutic Efficacy of a Synthetic Defensin Against Infective Endocarditis (IE) Caused by Methicillin-Resistant Staphylococcus aureus (MRSA)

### Methods

### Minimum Inhibitory Concentrations (MICs)

The MICs of endocardisin, vancomycin and daptomycin were determined by a broth microdilution assay according to Clinical Laboratory Standards Institute (CLSI). MIC is defined as the lowest drug concentration completely inhibiting organisms growth.

### Animal Endocarditis Model

Experimental aortic infective endocarditis model was induced in New Zealand White rabbits with a methicillin-resistant Staphylococcus aureus (MRSA), strain ATCC33591, following trans-carotid-transaortic valve indwelling catheterization.

### 1) The Intrinsic Virulence of MRSA ATCC33591 in the IE Model

At 24 hours after catheterization, aortic-catheterized animals were challenged intravenously with 10⁵, 10⁶, or 10⁷ colony forming units (CFU) of the MRSA ATCC33591 strain per animal, the inoculum range that encompasses the ID₉₅ for most S. aureus strains in this model. Twenty-four hours after inoculation, all animals were euthanized, and their cardiac vegetations, kidneys, and spleen were removed and quantitatively cultured.

### 2) The Efficacy of Antimicrobial Agent Treatments

After aortic catheterization and 24 hr post-infection, animals were randomized to receive either:

i) no therapy (control);
ii) endocardisin at 40 mg/kg, iv, twice daily;
iii) vancomycin at 15 mg/kg, iv, twice daily; or
iv) daptomycin at 12 mg/kg, iv, once daily (the latter two dose-strategies to mimic human-like PK).

Treatments lasted for 3 days. At 24 hours after the last antibiotic dose, half the animals were sacrificed with a lethal dose of sodium pentobarbital to test the treatment efficacies. The remaining animals resided drug-free for an extra 3 days for relapse studies. At sacrifice, vegetations, kidneys and spleen were removed and quantitatively cultured. To monitor in vivo resistance development, homogenate tissues were cultured in parallel on TSB agar plates containing endocardisin (1×MIC=0.5 micrograms/ml). Culture results were expressed as mean log₁₀ CFU per gram of tissue (±SD).

### Results

### Minimum Inhibitory Concentrations (MIC)

The MICs of endocardisin, vancomycin and daptomycin for the study strain (MRSA ATCC33591) were 0.5, 2.0 and 0.125 micrograms/ml, respectively.

### The Intrinsic Virulence of MRSA ATCC33591 in the IE Model

At the 10⁵ CFU challenge, only 60% of catheterized animals developed infective endocarditis. At inocula of 10⁶ CFU and 10⁷ CFU challenge, all catheterized animals developed IE (Table 1). However, ˜33% of animals infected with the MRSA ATCC33591 strain at an inoculum of 10⁷ CFU died 5 24 hours post infection. Therefore, 10⁶ CFU was chosen for the following efficacy studies.

### The Efficacy of Antimicrobial Agent Treatments

MRSA densities in the different therapy and relapse groups are shown in Table 2. Therapy with all three antimicrobial agents significantly reduced MRSA densities in all three target tissues at the end-of-treatment as compared with untreated controls (Table 2; P<0.002). In addition, endocardisin treatment showed a significantly greater efficacy in reduction of MRSA densities in all target tissues as compared with vancomycin therapy (Table 2; P<0.001). Daptomycin had similar therapeutic efficacy in the IE model as compared with endocardisin (Table 2). Of importance, the endocardisin regimen was most effective in preventing relapse (Table 3). Moreover, no endocardisin-resistant strains were isolated (data not shown).

### Conclusions

These results confirm that endocardisin had significantly better efficacy in decreasing MRSA densities in all three target tissues as compared with the vancomycin treatment group, and had similar therapeutic efficacy as compared with the daptomycin regiment in a severe model of MRSA IE.

TABLE 1
Staphylococcus aureus densities in target tissues.

| | Staphylococcus aureus densities | | |
|---|---|---|---|
| | (log₁₀ CFU/g tissue) | | |
| Infection inoculum | Vegetations | Kidneys | Spleen |
| 10⁵ CFU | 6.51 +/− 3.61 | 4.02 +/− 2.72 | 3.85 +/− 2.05 |
| 10⁶ CFU | 8.08 +/− 0.85 | 6.13 +/− 0.88 | 5.49 +/− 0.43 |
| 10⁷ CFU | 8.83 +/− 0.91 | 6.46 +/− 0.77 | 5.90 +/− 0.51 |

TABLE 2
Efficacies of endocardisin, vancomycin and daptomycin against
MRSA ATCC33591 in an experimental rabbit endocarditis model.

| | Staphylococcus aureus densities | | |
|---|---|---|---|
| | (log₁₀ CFU/g tissue) | | |
| Treatment | Vegetations | Kidneys | Spleen |
| Control without treatment | 8.08 +/− 0.85 | 6.13 +/− 0.88 | 5.49 +/− 0.43 |
| Endocardisin | 2.41 +/− 1.50* | 1.18 +/− 0.75* | 1.02 +/− 0.65* |
| (40 mg/kg, iv, twice-daily) | | | |
| Vancomycin | 5.60 +/− 1.47 | 3.07 +/− 0.53 | 3.02 +/− 0.51 |
| (15 mg/kg, iv, twice-daily) | | | |
| Daptomycin | 1.90 +/− 0.82* | 0.76 +/− 0.52* | 0.91 +/− 0.53* |
| (12 mg/kg, iv, once-daily) | | | |

*P < 0.001 vs. vancomycin treatment.

TABLE 3
Efficacies of endocardisin, vancomycin and daptomycin against
MRSA ATCC33591 in an experimental rabbit endocarditis model.

| | Staphylococcus aureus densities | | |
|---|---|---|---|
| | (log₁₀ CFU/g tissue) | | |
| Relapse | Vegetations | Kidneys | Spleen |
| Endocardisin | 1.45 +/− 0.64* | 0.75 +/− 0.79* | 1.17 +/− 0.70* |
| (40 mg/kg, iv, twice-daily) | | | |
| Vancomycin | 6.36 +/− 1.57 | 4.69 +/− 1.63 | 3.98 +/− 1.27 |
| (15 mg/kg, iv, twice-daily) | | | |
| Daptomycin | 2.46 +/− 2.36** | 1.68 +/− 2.24** | 1.30 +/− 1.48** |
| (12 mg/kg, iv, once-daily) | | | |

*P < 0.001 vs. vancomycin relapse;
**P < 0.01 vs. vancomycin relapse.

### Example 2

### Therapeutic Efficacy of a Synthetic Defensin Against Infective Endocarditis (IE)

### Methods

Rabbit IE was induced following transcarotid-transaortic valve indwelling catheterization. At 24 hrs after iv infection with 10⁶ cfu MRSA ATCC33591 (ID₉₅ inoculum), animals received either:

i) no therapy (control);
ii) endocardisin at 5, 10 or 20 mg/kg, iv, bid;
iii) vancomycin at 15 mg/kg, iv, bid; or
iv) daptomycin at 12 mg/kg, iv, once-daily;

All regimens were for 3 days. At 24 hours after the last antibiotic dose, target tissues were removed and quantitatively cultured.

### Results

Each regimen significantly decreased MRSA densities in three major target tissues vs. untreated controls (P<0.05), except the lowest dose of endocardisin in reduction of splenic MRSA counts. In addition, a dose-dependent therapeutic efficacy of endocardisin was observed in all target tissues in the IE model. Importantly, endocardisin at 20 mg/kg showed a significantly greater efficacy in reduction of MRSA densities in all target tissues vs. vancomycin therapy, and similar efficacy to daptomycin.

TABLE 4
Efficacies of endocardisin, vancomycin and daptomycin against
MRSA ATCC33591 in an experimental rabbit endocarditis model.

| | Staphylococcus aureus densities | | |
|---|---|---|---|
| | (log₁₀ CFU/g tissue) | | |
| Treatment | Vegetations | Kidneys | Spleen |
| Control without treatment | 8.15 +/− 0.73 | 5.82 +/− 0.85 | 5.28 +/− 0.55 |
| Endocardisin | 1.57 +/− 0.75 | 0.61 +/− 0.21 | 0.61 +/− 0.12 |
| (20 mg/kg, iv, twice-daily) | | | |
| Endocardisin | 4.74 +/− 1.84 | 3.19 +/− 1.06 | 2.62 +/− 0.60 |
| (10 mg/kg, iv, twice-daily) | | | |
| Endocardisin | 6.44 +/− 1.70 | 4.33 +/− 1.27 | 4.61 +/− 1.11 |
| (5 mg/kg, iv, twice-daily) | | | |
| Vancomycin | 5.60 +/− 1.47 | 3.07 +/− 0.53 | 3.02 +/− 0.51 |
| (15 mg/kg, iv, twice-daily) | | | |
| Daptomycin | 1.90 +/− 0.82 | 0.76 +/− 0.53 | 0.91 +/− 0.53 |
| (12 mg/kg, iv, once-daily) | | | |

### Conclusions

The superior efficacy of endocardicin vs. vancomcyin, and equivalent efficacy to daptomycin in this model of severe multisystem MRSA infection suggest the potential for further development of this compound for treating clinical syndromes.

1-17. (canceled)

18. A method of treating infective endocarditis, comprising administering to a subject in need of such treatment an effective amount of a polypeptide having antimicrobial activity, which comprises an amino acid sequence having at least 80% identity to the amino acid sequence of SEQ ID NO: 1.

19. The method of claim 18, wherein the polypeptide comprises an amino acid sequence having at least 90% identity to the amino acid sequence of SEQ ID NO: 1.

20. The method of claim 18, wherein the polypeptide comprises or consists of the amino acid sequence of SEQ ID NO: 1.

21. The method of claim 18, wherein the infective endocarditis is caused by a Staphylococcus sp.

22. The method of claim 18, wherein the infective endocarditis is caused by Staphylococcus aureus.

23. The method of claim 18, wherein the infective endocarditis is caused by a methicilin-resistant Staphylococcus aureus (MRSA).

24. A method for preventing relapse from a treatment of infective endocarditis, comprising administering to a subject in need of such treatment an effective amount of a polypeptide having antimicrobial activity, which comprises an amino acid sequence having at least 80% identity to the amino acid sequence of SEQ ID NO: 1.

25. The method of claim 24, wherein the polypeptide comprises an amino acid sequence having at least 90% identity to the amino acid sequence of SEQ ID NO: 1.

26. The method of claim 24, wherein the polypeptide comprises or consists of the amino acid sequence of SEQ ID NO: 1.

27. The method of claim 24, wherein the infective endocarditis is caused by a Staphylococcus sp.

28. The method of claim 24, wherein the infective endocarditis is caused by Staphylococcus aureus.

29. The method of claim 24, wherein the infective endocarditis is caused by a methicilin-resistant Staphylococcus aureus (MRSA).


SEQUENCE LISTING (1 sequence)

SEQ ID NO: 1 - protein, 40 aa, Artificial sequence
  Synthetic construct
GFGCNGPWNEDDLRCHNHCKSIKGYKGGYCAKGGFVCKCY